(19) United States
(12) Patent Application Publication
(10) Pub. No.: US 2005/0004472 A1
(43) Pub. Date: Jan. 6, 2005

(54) Medical socket contour scanning system

(76) Inventor: Greg Pratt, Boca Raton, FL (US)

Correspondence Address: MALLOY & MALLOY, P.A., Historic Coral Way, 2800 S.W. Third Avenue, Miami, FL, 33129, US

(21) Appl. No.: 10/641,895

(22) Filed: Aug. 15, 2003

Related U.S. Application Data
(60) Provisional application No. 60/404,209, filed on Aug. 17, 2002.

(51) Int. Cl. A61B 6/00

(52) U.S. Cl. 600/476

(57) ABSTRACT

To scan a body part of a patient, a medical socket contour scanning system including a pattern imposer that imposes a predefined pattern having a plurality of contrast points on the body part to be scanned, and a scanning element including a plurality of image capture devices disposed a pre-defined, spaced apart distance from one another such that adjacent ones have an overlapping field of view. The image capture devices are structured to simultaneously and instantaneously capture an image of the body part with the pattern imposed thereon, a processor, operatively associated with the image capture devices structured to receive the captured images from the image capture devices, compare the captured images from adjacent ones of the image capture devices in order to identify a common element of the pattern, and to utilize the common element so as to define a three-dimensional composite image from all of the captured images which will be utilized to define a socket for a medical appliance.

### CLAIM OF PRIORITY

The present application is based on and a claim to priority is made under 35 U.S.C. Section 119(e) to provisional patent application currently pending in the U.S. Patent and Trademark Office having Ser. No. 60/404,209 and a filing date of Aug. 17, 2002.

### BACKGROUND OF THE INVENTION

1. Field of the Invention

The present invention relates to a scanning system which can be utilized to scan a medical socket contour from a body part, such as a patient's limb, head, torso, etc. so as to facilitate the production of an orthotic or prosthetic appliance, such as an artificial limb, leg, knee or foot brace, molded cranial helmet, spinal brace, etc. which includes a socket that conforms to the socket contour defined by the body part to which it will be secured. In particular, the scanning system imposes an identifiable reference pattern on the body part to be scanned, and instantaneously obtains a substantially complete and detailed three dimensional scan of the body part, which can be effectively processed in order to generate a composite image from which the socket contour can be defined. Using the socket contour, an appropriate medical appliance can be molded to a substantial degree of accuracy without significantly inconveniencing the patient.

2. Description of the Related Art

The fields of orthotic and prosthetic medicine are very specialized and is continuously making technological advances. One factor, however, that remains constant within these medical arts relates to the need for a precise fit between the medical appliance and the patient for which it will be fitted. For example, in the field of artificial limbs, such as artificial legs or arms, an artificial leg or arm will typically include a socket into which the distal portions of the patient's limb will be introduced. Naturally, if a substantially exacting and/or appropriate fit is not achieved, a significant amount of patient discomfort can result and/or a malfunction of the orthotic appliance can result. Similarly, another advancing field of medicine relates to the formation of molded cranial helmets for infants or small children. Specifically, these cranial helmets must be effectively fitted and contoured to match the patient's skull, while still maintaining an appreciable degree of comfort, especially considering the inability of the child to communicate effectively. Accordingly, it can be seen that in a variety of different fields of orthotic and prosthetic medicine, there is a substantial need to effectively map or identify the body part which will come into contact and/or otherwise engage the medical appliance.

Traditionally, the appropriate fit for the medical appliance has been achieved through the utilization of plaster or other type casting materials. For example, a limb or other body part to be joined with the medical appliance is covered with a corresponding molding material such that a mold or cast of that body part can be generated. This cast is then used so as to create a corresponding male cast element about which the socket for the medical appliance can be generated. As can be appreciated, however, such casting procedures are often somewhat imprecise and/or inconvenient to utilize, and leave little room for modification and/or manipulation by a practitioner in order to provide a comfort fit. For example, these casting procedures are by their nature substantially messy, time consuming and inconvenient. As a result, specialized facilities must often be employed in order to effectively make a mold or cast of the patient's body part. Additionally, however, the formation of these casts often requires that the body part be maintained substantially still and/or covered while the plaster material is being applied, a procedure that can be substantially difficult for a patient, and in particular a small child who may not sufficiently understand the need to keep still. Furthermore, practitioners have over the years learned that slight modifications and/or adjustments to the exact contour of the patient's body may be necessary in order to relieve pressure points and/or otherwise provide for a more appropriate fit. The use of a typical cast molding system, however, requires that the practitioner operate by feel, after the cast has been made, in order to carve out or otherwise form modifications or alterations. Naturally, such carving out type procedures can be imprecise and difficult to effectively manage and manipulate, usually requiring a degree of skill and/or artistry only found in very experience practitioners. As a result, there is a substantial need in the art for a scanning system which will eliminate the need to utilize plaster or other type casting materials and which will provide practitioners a substantial ease of use, even if they are generally inexperienced, and will provide a substantial degree of adaptability in order to meet the specific needs of the patient and/or the medical appliance.

In addition to the casting systems, it is also recognized that others in the art have also turned to digital or electronic type scanning systems in order to effectively achieve a computer generated image of the body part. Unfortunately, however, typical scanning devices have substantial drawbacks associated therewith, those drawbacks often associated with the need for the body part to be maintained completely still during the entire scanning procedure. For example, many scanning systems incorporate the use of cameras and/or lasers and/or a combination thereof to identify and/or map contours in an object to generate a three dimensional scan. Such systems, however, are usually not designed for scanning animate objects and can often be substantially complex and expensive to implement and utilized. Furthermore, even the fastest system require, at a minimum, one to two seconds of scanning time in order to effectively generate an image, a period of time that can be too long if an accurate scan is truly to be obtain. In particular, and looking again to the field of molded cranial helmets, it is substantially difficult if not impossible to effectively maintain an infant's or small child's head completely stationary for even just a few seconds. As a result, when using a typical system, a generally imprecise scan of the body part will typically result. Furthermore, it is recognized that with such systems the body part being scanned must generally be maintained in a suspended orientation such that effective and complete scanning thereof can be achieved. Such suspension of the body part further leads to the difficulty associated with maintaining the body part still and in a continuous position and orientation while the complete scan is taken. As can be realized, if movement results the scan will no longer achieve the precision required in order to achieve effective socket formation. Also, it is recognized that even with those systems that utilize a laser so as to map out a grid on the body part to be scanned, such light or laser based scanning devices can significantly lose their accuracy as the body part of the patient moves there under. As a result, such scanning systems are typically designed for and limited to the scanning of inanimate objects. Accordingly, there is also a substantial need in the art for a scanning system which can effectively provide a three dimensional composite image of a body part of a patient to be scanned, can work very quickly and conveniently, not requiring the body part to remain perfectly still or suspended for an extended period of time, and allowing for movement while still substantially quickly achieving the precise scan that is required. Furthermore, there is a need for such system which will not require complete reorientation, calibration, and/or positioning of the system when used with animate objects which inherently move.

### SUMMARY OF THE INVENTION

The present invention relates to a medical socket contour scanning system utilized to scan a body part of a patient. In particular, the scanning system includes a pattern imposer. The pattern imposer is structured to impose a pattern having a plurality of contrast points onto the body part to be scanned. Furthermore, this imposed pattern is preferably structured to move with the body part, and to substantially conform to the contours of the body part.

Additionally, the present scanning system includes a scanning element. The scanning element includes a plurality of image capture devices disposed a predefined, spaced apart distance from one another. These image capture devices are positioned such that adjacent ones thereof have an at least partially overlapping field of view. Furthermore, the image capture devices are structured to substantially simultaneously and instantaneously capture an image of the body part that has the pattern imposed thereon, the captured images obtained by adjacent ones of the image capture devices also necessarily overlapping one another.

A processor is operatively associated with the image capture devices and is structured to receive the captured images therefrom. The processor is also structured to compare the captured images from at least two adjacent ones of the image capture devices in order to identify a common element of the pattern in each of the adjacent captured images. The processor can then utilize that common element in each of the adjacent captured images so as to define a three dimensional composite image from the adjacent captured images. In the preferred embodiment, all of the captured images from the plurality of image capture devices can be effectively linked to their adjacent images and a complete three dimensional composite image, including an image scanning approximately 180 degrees around the body part and/or preferably 360 degrees around the body part, can be generated. Naturally, utilizing the three dimensional composite image, and possibly some manipulation and/or adjustment thereof, an appropriate socket for a medical appliance can be constructed.

These and other features and advantages of the present invention will become more clear when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a an illustration of a preferred embodiment of the scanning system of the present invention, and in particular, the scanning element and processor thereof;

FIG. 2 is an illustration of a preferred embodiment of the pattern imposer disposed on the head of a patient as part of a cranial scan;

FIG. 3 is an exploded view of a preferred embodiment of the pattern imposer to be applied to a limb of a patient as the body part to be scanned;

FIG. 4 is an illustration of an alternative embodiment of the pattern imposer;

FIG. 5 is an illustration of an alternative embodiment of the scanning element which is preferably utilized in connection with the scanning of a torso;

FIG. 6 is an illustration one medical appliance which may be manufactured utilizing the scanning system of the present invention;

FIG. 7 is a perspective illustration use of one embodiment of the alignment assembly of the present invention; and

FIG. 8 is a side plan view of another embodiment of the alignment assembly of the present invention.

Like reference numerals refer to like parts throughout the several views of the drawings.

### DETAILED DESCRIPTION OF THE PREFERRED EMBODIMENT

As shown throughout the Figures, the present invention is directed towards a medical socket contour scanning system, generally indicated as 10. In particular, the scanning system 10 is structured to scan a body part 70 of a patient in order to effectively produce a three dimensional composite image, which in the preferred embodiment, can be manipulated and adjusted in order to precisely define a socket contour from which a socket 76 for a medical appliance, such as an artificial limb 75, can ultimately be formed. It is the socket 76 which will generally be fitted into appropriate engaging relation with a corresponding body part of the patient, and as a result must substantially conform to that body part and/or the specifications of a practitioner for appropriate use and implementation.

Looking to the Figures, the scanning system 10 preferably includes a pattern imposer, generally 20. The pattern imposer is structured to impose a specific pattern 26 having a plurality of contrast point on the body part to be scanned. As such, the pattern 26 defined by the pattern imposer 20 provides for effective identification and appropriate three dimensional imaging, as will be described, of the specific body part.

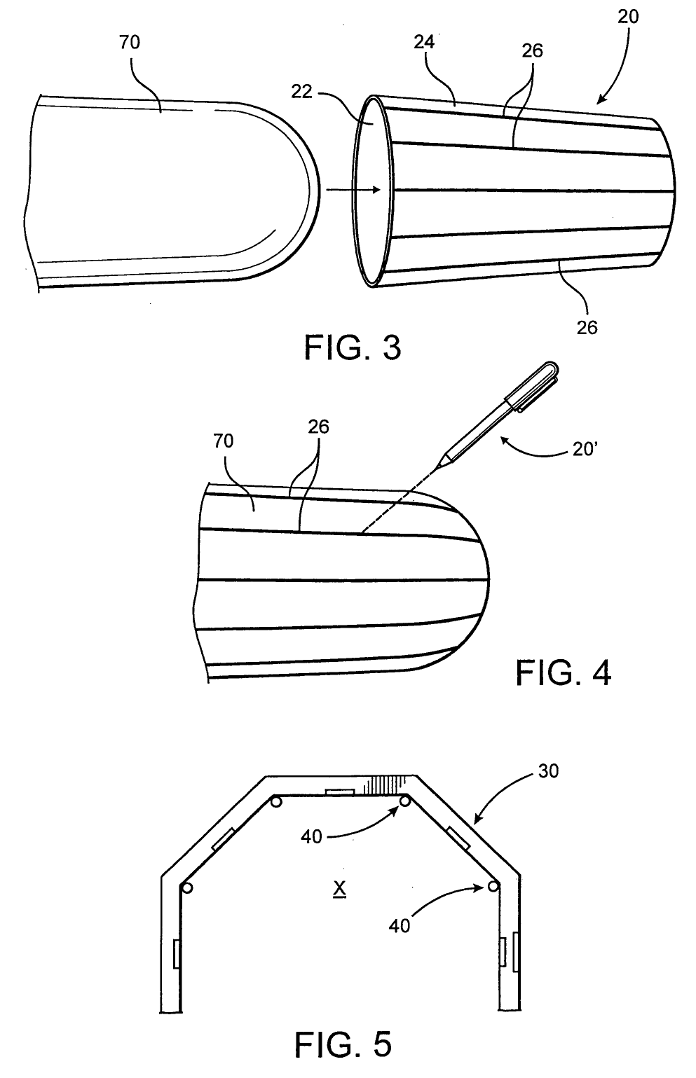

Looking to the illustrated embodiments, the pattern 26 is defined by a series of stripes, which define a substantially uniform set of contrast points, and is positioned on the body part 70 by the pattern imposer 20 in such a manner that it will move with the body part 70 and will substantially conform to the contours of the body part 70. Moreover, in the first illustrated embodiments of FIGS. 2 and 3, wherein the patient's head or limb extremity is the body part 70 to be scanned, the pattern imposer 20 preferably comprises a covering element 24 which includes the pattern 26 disposed thereon. Specifically, the covering element 24 is preferably formed from an at least partially elastic and/or resilient material that will substantially conform to the contours of the body part 70 to be scanned. As seen in FIG. 3, the covering element 24 may include a material sleeve which has an opening 22 into which the body part 70 is disposed. As a result, the elastic properties of the covering element 24 effectively ensure that substantial conformity and/or form fitting to the body part 70 can be achieved. Indeed, for this reason, it is often preferred that the covering element 24 be of a somewhat smaller size that must stretch in order to effectively fit over the body part 70.

Looking to FIG. 4, in an alternative, less preferred embodiment, the pattern imposer 20′ may include a marking element which actually forms, such as by drawing, the pattern 26 directly on the skin of the body part 70. Nevertheless, in either embodiment, the pattern 26 defines a series of contrast points that can be effectively utilized in the scanning process. As indicated, in the illustrated embodiment the pattern 26 is defined by a series of spaced apart stripes, for example black stripes on a white background. Of course, however, it is understood that other colors and/or other patterns, whether uniform or varied, may also be effectively utilized to define the necessary contrast points.

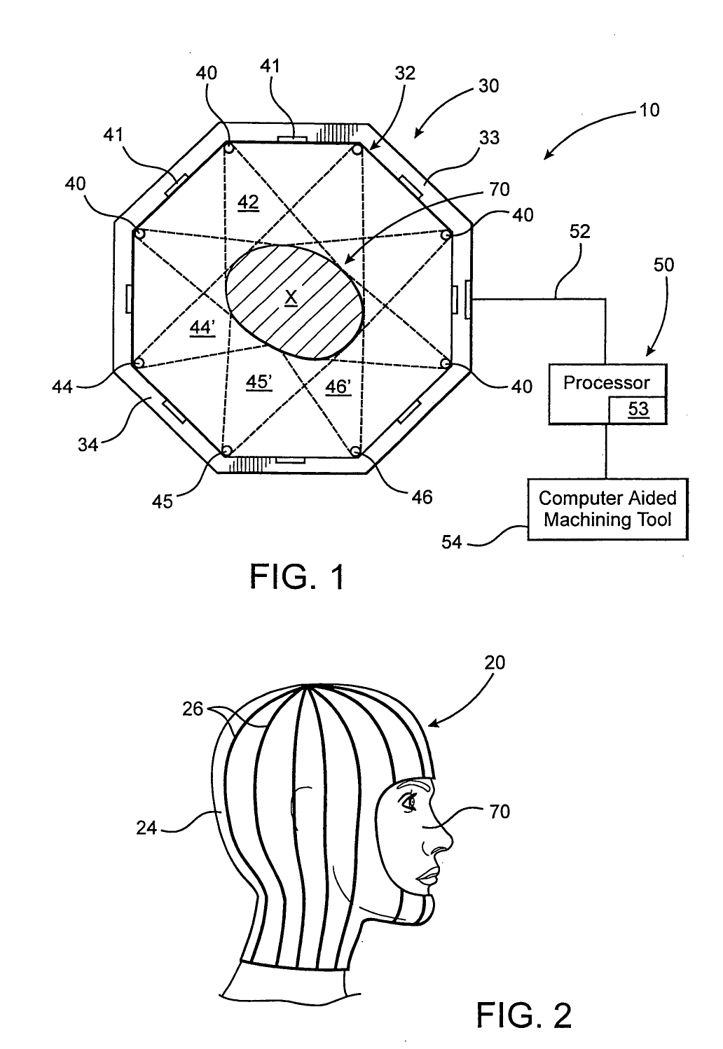

In addition to the pattern imposer 20, the present scanning system includes a scanning element, generally indicated as 30. The scanning element 30 preferably includes a plurality of image capture devices 40, disposed of predefined spaced apart distance from one another, and structured to capture images of the body part 70 being scanned. In the illustrated embodiments, the scanning element 30 includes at least one rigid support 32 on which the image capture devices 40 may be mounted in their predefined spaced apart relation from one another. The rigid support is preferably provided so as to provide for facilitated manipulation and uniform positioning of the image capture devices 40, and as in the embodiment of FIG. 1, may include a ring or other frame type of configuration that partially or completely surrounds the body part 70. In this regard, it is noted that the rigid support 32 may be partially open so long as it positions the image capture devices 40 in their appropriate, spaced apart configuration from one another, with a sufficient field of view for image capturing.

The images captured devices 40 of the present invention are preferably disposed such that their field of views at least partially overlap one another, and such that they are positioned and/or angled towards a substantially common axis. Furthermore, in the embodiments of FIG. 1, the image capture devices 40 are preferably positioned so as to substantially surround the body part 70 which will be scanned. In this regard, it is again noted that even in the embodiment of FIG. 1 the rigid support 32 need not necessarily completely surround the body part 70 so long as the image capture devices 40 are positioned to essentially surround the body part 70 based upon their field of view's coverage of the body part 70. For example, a generally U-shaped rigid support 32 and/or a pair of spaced apart substantially straight or angled bars may also be utilized.

When scanning of the body part 70 is to be performed, the body part 70 must necessarily be within the field of view of at least some, and preferably all of the image capture devices 40. As such, the body part 70 is preferably generally centered between the image capture devices 40 in an aligned orientation, as will be described subsequently with regard to the alignment assembly 80. In the embodiment of FIG. 1, the rigid support 32 may be passed over the extremity of the body part 70, however, if desired, the rigid support 32 may include a pair of support elements 33 and 34 hingedly secured to one another so as to effectively allow them to surround the body part 70 of the patient when the portion to be scanned is not sufficiently at the extremity so as to allow for effective passage of the body part 70 into the scanning area. Moreover, transportability and/or storability is also aided by the ability to open and/or collapse the rigid support 32.

Although in the preferred embodiments the image capture devices 40 will be positioned such that their field of views substantially surround the body part 70 to be scanned, in an alternative embodiment illustrated in FIG. 5, it is recognized that the condition of the patient may not necessarily allow for effective suspension of the body part 70 in an orientation whereby it can be surrounded by image capture devices 40. An example of such a circumstance relates to when a spinal and/or body brace must be made and the portion of the body being scanned includes the torso of the patient. In such a circumstance, it may be beneficial to achieve one or two scans of the body part to ultimately define the medical socket contour. In such an embodiment, the scanning element 30′ may include an open configuration, such as for example only one of the support elements 33 or 34, with the image capture devices 40 positioned such that their field of view covers at least 180 degrees of the body part 70 to be scanned.

As indicated, preferably a plurality of image capture devices 40 are provided for use in the present scanning system. In a 360 degree scan embodiment it is preferred that eight image capture devices be used, whereas in a 180 degree scan embodiment it is preferred that six image capture devices 40 be used. Of course, this number can vary depending, for example, upon the field of view of the image capture devices. Looking to the image capture devices 40 themselves, preferably a plurality of digital or ccd cameras are utilized, and preferably a black and white or monochrome image is captured so as to maximize the detailed, contrast, and clarity of the captured image. Of course, it is understood that a variety of different image capture devices 40 may be employed so long as a substantially high quality and detailed image of its field of view can be captured thereby.

In order to maximize the quality and accuracy of the captured image, in addition to the image capture devices 40 being disposed a predetermined spaced apart distance from one another within a rigid frame, a calibration assembly may also be incorporated. For example, a series of calibration elements 41, such as LEDs and/or other markings, illuminations, and/or reference points may be provided at a known position. As a result, by having each image captured device 40 capture an image containing those known calibration elements 41, a highly accurate determination of the relative orientation and positioning of each of the image capture devices 40 may be achieved and retained for use in defining a composite image. In this regard, it is also noted that the calibration assembly need not be directly associated with and/or mounted on the scanning element 30. It is also noted that transportability and/or storability is aided by incorporating the calibration elements directly in the scanning element 30

In addition to capturing images of the body part 70 with at least partially overlapping fields of view, the image capture devices 40 are further structured to substantially simultaneously and instantaneously capture those images of the body part having the pattern 26 imposed thereon. Accordingly, even slight movement by the body part 70 will not result in a distortion and/or ultimate misalignment of the images captured by each of the image capture devices 40. This is particularly important when the body part 70 to be scanned is the head of a patient, such as an infant or small child. For example, when making a cranial scan for formation of a cranial helmet, it is often impossible to maintain a child's head stationary for even a very brief period of time. As such, based upon the fact that the image capture devices 40 of the present invention are structured to substantially simultaneously and instantaneously capture the images, slight movements will not affect the relative positioning and/or orientation of the overlapping images captured by each of the image capture devices 40.

Further included as part of the present invention is a processor 50. In particular, the processor 50 may include a separate processor, as in the illustrated embodiment and including, for example, a computer connected via a cable 52 and/or a wireless connection, may include one or more processors directly associated with each of the image capture devices 40 and/or may include a combination thereof. The processor 50 preferably helps to coordinate the simultaneous image capture from each of the image capture devices 40. Moreover, once the images have been captured, the processor 50 is structured to compare captured images from the fields of view 44′ and 45′ of at least two adjacent ones of the image capture devices 44 and 45. In comparing those adjacent images, the processor 50 is structured to identify at least one common element of the pattern 26 that has been captured in each of the adjacent captured images. In order to facilitate this process, in one embodiment of the present invention a specific reference point, such as any identifiable deviation and/or marking on the pattern 26 can be defined by the pattern imposer within the at least the two, initially compared, adjacent captured images. As such, the reference point can function as the common element identified by the processor 50 for the adjacent captured images. Alternatively, however, and/or in addition to the utilization of the predefined reference point in the pattern 26, in another embodiment of the present invention the processor 50 may utilize the captured image in the field of view 46′ of yet another subsequently adjacent image capture device 46. By using this third captured image a triangulation function can be performed for the purposes of defining the common element. Once the common element between the two adjacent captured images has been identified, the processor 50 is then able to define a three dimensional composite image from all of the capture images. For example, the processor 50 may in essence stitch the adjacent images with one another, utilizing the pattern 26 that has been imposed on the body part 70 as a reference for overlapping and/or stitching the images. Moreover, once the initial common element between the first two adjacent captured images has been identified, the nature of the pattern 26 allows for substantially facilitated overlapping and/or stitching of the next adjacent captured image until the entire three dimensional composite image has been appropriate defined.

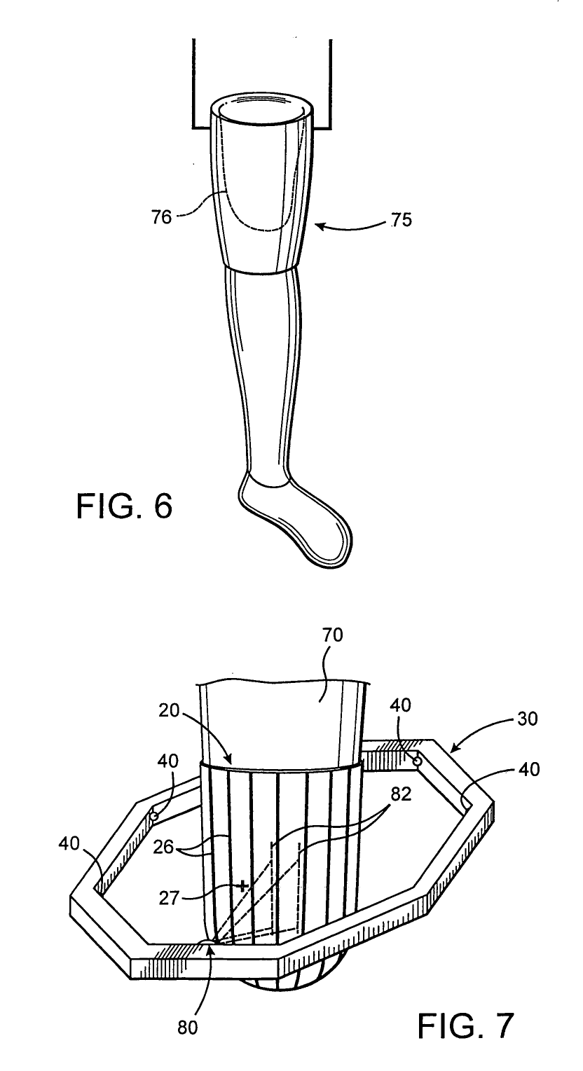

The processor 50, either independently and/or in association with a further computer processor and/or machining device, such as a computer aided machining tool represented schematically as 54, is further structured to define a medical socket contour for the medical appliance, the socket contour corresponding to the three dimensional composite image that has been achieved utilizing the overlapped captured images. Also, in the preferred embodiment a user interface 53, such as in the form of a key pad, pointer control and or other input device structured to receive user inputs, is provided, and as such, the socket contour may be modified to meet the true needs of the patient as defined by a practitioner. For example, and with reference to FIG. 6, wherein the medical appliance includes an artificial limb 75, it is often preferred that the socket contour not exactly match the exterior contours of the body part 70 which will be fitted therein. As a result, using the user interface 53 of the processor 50, modifications can be made to the socket contour that was defined utilizing the three dimensional composite image, those contours providing necessary gaps, supports and/or recesses to increase the comfort, therapeutic benefit and/or usability of the medical appliance for its required purpose. With the finalized and/or desired socket contour defined, data and/or information which defines the socket contour can be provided to a conventional computer aided machining tool so as to ultimately define an actual socket 76 of the medical appliance 75 which precisely matches the socket contour 70 that was defined utilizing the present invention. As indicated, the present scanning system may be utilized in connection with the construction of a plurality of different medical appliances, and as such, the socket contour may be utilized so as to define a socket for a spinal brace, a limb brace, such as a prosthetic appliance including a prosthetic socket or prosthetic limb, a cranial helmet, an artificial limb and/or any other medical appliance that needs to have a precise contour matching a corresponding body part 70 or a patient on which it will be utilized.

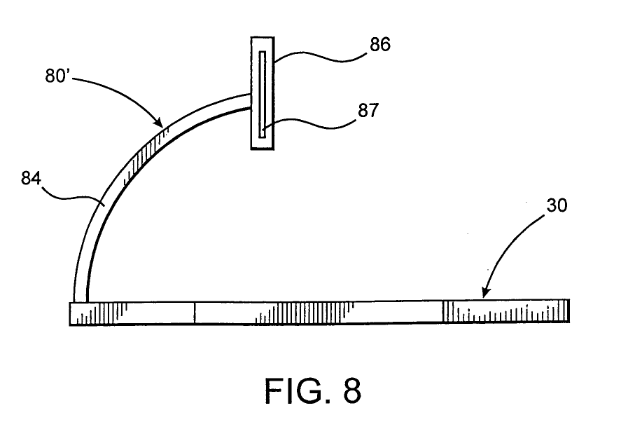

With reference to FIGS. 7 and 8, it is also noted that the present invention may also include an alignment assembly 80. Specifically, the alignment assembly 80 is structured to preferably position the body part 70 in an appropriate scanning position and orientation, such as for example at the convergent axis of the fields of view 42 of all the image capture devices 40, and/or in appropriate vertical alignment. In one embodiment of the alignment assembly 80, as illustrated in FIG. 7, a light emitter, such as a laser light emitter is structured to emit a pattern of light 82 to be projected on the body part, and preferably on the pattern 26 of the pattern imposer. By aligning the pattern of light 82 with the pattern 26 defined by the pattern imposer, a practitioner can be substantially assured that the body part is substantially in an appropriate scanning position and orientation. As an alternative embodiment, the alignment assembly 80′ may include a support arm 84 that extends from the scanning element 30 and positions an alignment member 86. The body part 70 to be scanned can then be positioned in engagement with the alignment member, thus substantially ensuring it is appropriately positioned and aligned. Furthermore, if desired, an alignment indicia 87 may be disposed on the alignment member 86 to further achieve appropriate alignment.

Also, as a further point of reference, it may be desirable for a practitioner to identify one or more landmarks 27 in proximity to the the pattern 26. This landmark 27 can be pre-placed relative to the pattern 26 or can be defined manually be a practitioner, such as utilizing a marking pen or tape. Accordingly, when the three-dimensional composite image is defined, the landmark 27 can also be identified and utilized as an effective reference point, such as in connection with needed shape modifications or to set an anatomical landmark.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

1. To scan a body part of a patient, a medical socket contour scanning system comprising:
a) a pattern imposer, said pattern imposer structured to impose a pattern having a plurality of contrast points on the body part to be scanned;
b) a scanning element, said scanning element including a plurality of image capture devices disposed a pre-defined, spaced apart distance from one another;
c) said image capture devices disposed such that adjacent ones have an at least partially overlapping field of view;
d) said image capture devices structured to substantially simultaneously and instantaneously capture an image of the body part with said pattern imposed thereon;
e) a processor structured to receive said captured images from said image capture devices;
f) said processor structured to compare said captured images from at least two adjacent ones of said image capture devices and to identify a common element of said pattern in each of said adjacent captured images; and
g) said processor structured to utilize said common element in each of said adjacent captured images so as to define a three-dimensional composite image from said adjacent captured images.

2. A scanning system as recited in claim 1 wherein said pattern is structured to move with the body part.

3. A scanning system as recited in claim 1 wherein said pattern is structured to substantially conform to contours of the body part of the patient.

4. A scanning system as recited in claim 1 wherein said pattern imposer comprises a marking element structured to mark the body part so as to impose said pattern on the body part.

5. A scanning system as recited in claim 1 wherein said pattern imposer comprises a covering element structured to be positioned on the body part in substantially conforming relation thereto.

6. A scanning system as recited in claim 5 wherein said covering element includes said pattern disposed thereon.

7. A scanning system as recited in claim 5 wherein said covering element is formed from an elastic material.

8. A scanning system as recited in claim 1 wherein said pattern imposer comprises an elastic material sleeve structured to be disposed on the body part to be scanned in substantially form fitting relation thereto, said elastic material sleeve including indicia thereon structured to define said pattern.

9. A scanning system as recited in claim 1 wherein said scanning element includes at least one rigid support on which said image capture devices are disposed in a spaced apart relation from one another.

10. A scanning system as recited in claim 1 wherein said scanning element comprises a rigid support on which said image capture devices are disposed in spaced apart relation from one another, said rigid support structured to position said image capture devices generally towards a common axis and in position to cover at least a 180 degree area.

11. A scanning system as recited in claim 1 wherein said scanning element comprises a rigid support on which said image capture devices are disposed in spaced apart relation from one another and angled towards a substantially common axis.

12. A scanning system as recited in claim 11 wherein said rigid support is structured to surround the body part and position said image capture devices generally towards a common axis.

13. A scanning system as recited in claim 1 wherein said plurality of contrast points of said pattern include a plurality of stripes.

14. A scanning system as recited in claim 1 wherein said pattern further includes at least one predefined reference point.

15. A scanning system as recited in claim 1 wherein said image capture devices are structured to capture a monochrome image.

16. A scanning system as recited in claim 1 comprising eight of said image capture devices disposed in surrounding relation to the body part.

17. A scanning system as recited in claim 1 comprising six of said image capture devices disposed in surrounding relation to the body part.

18. A scanning system as recited in claim 1 wherein said processor assembly is structured to define a socket contour for a medical appliance which corresponds to said three dimensional composite image.

19. A scanning system as recited in claim 18 wherein said processor assembly includes a user interface, said user interface configured to receive user inputs and to modify said socket contour in accordance with said user inputs.

20. A scanning system as recited in claim 19 wherein said processor assembly is structured to provide said socket contour data to a computer aided machining tool.

21. A scanning system as recited in claim 18 wherein said medical appliance includes a cranial helmet.

22. A scanning system as recited in claim 18 wherein said medical appliance includes a spinal brace.

23. A scanning system as recited in claim 18 wherein said medical appliance includes a limb brace.

24. A scanning system as recited in claim 23 wherein said limb brace comprises a prosthetic appliance.

25. A scanning system as recited in claim 1 further comprising an alignment assembly structured to facilitate appropriate positioning and orientation of the body part in said field of view of said image capture devices.

26. A scanning system as recited in claim 1 further comprising a calibration assembly structured to facilitate determination of a precise position and orientation of said image capture devices.

27. A scanning system as recited in claim 26 wherein said calibration assembly comprises a plurality of calibration elements whose positions are predefined, said image capture devices structured to capture a calibration image containing said calibration elements.

28. A scanning system as recited in claim 27 wherein said scanning element, including said calibration elements, is portable.

29. A scanning system as recited in claim 1 further comprising at least one landmark defined in operative proximity to said pattern defined by said pattern imposer and structured to define a reference point relative to the body part.

30. A scanning system as recited in claim 1 wherein said scanning element is portable.
